(19) United States
(12) Patent Application Publication
(10) Pub. No.: US 2014/0232989 A1
(43) Pub. Date: Aug. 21, 2014

(54) EYE FIXATION SYSTEM AND METHOD

(71) Applicant: THE JOHNS HOPKINS UNIVERSITY, Baltimore, MD (US)

(72) Inventors: Boris I. Gramatikov, Baltimore, MD (US); David L. Guyton, Baltimore, MD (US); Kristina Irsch, Baltimore, MD (US)

(73) Assignee: The Johns Hopkins University, Baltimore, MD (US)

(21) Appl. No.: 13/773,307

(22) Filed: Feb. 21, 2013

(51) Int. Cl. A61B 3/00

(52) U.S. Cl. CPC A61B 3/0091
USPC 351/211; 351/205; 351/246

(57) ABSTRACT

An eye fixation system for attention-attracting fixation of a user's eye includes an audio system configured to play sound based on audio signals to be audible to a user so as to attract the user's attention, and a target display system configured to communicate with the audio system to receive the audio signals and to display an optical target to be viewed by the user. The target display system includes an optical modulator configured to modulate the optical target based on the audio signals. A method of assisting a subject to fix at least one of his eyes in a predetermined alignment includes playing sound that is audible to the subject, displaying an optical target to be viewed by the subject synchronously with the sound such that the target is viewable by the subject while the sound is audible to the subject, and modulating the optical target based on the sound while the sound is being played.

### BACKGROUND

1. Field of Invention

The field of the currently claimed embodiments of this invention relates to eye fixation systems, and more particularly to eye fixations systems with combined audio and optical systems.

2. Discussion of Related Art

Many devices for eye diagnostics require the patient to fixate steadily on a small point in space for a certain period of time during which the eyes do not move and data from one or more substructures of one or both eyes are acquired and analyzed. Some diagnostic devices acquire data very fast, within less than a second, while others require tens of seconds. A typical example would be ophthalmic diagnostic devices for obtaining information from the retina, like a scanning laser ophthalmoscope, optical coherence tomography (OCT) devices, retinal tomographs, scanning laser polarimeters, retinal birefringence scanners, fundus cameras, and others. Other examples would be behavioral or psychological tests where deviations from steady fixation on a target are used as a differentiating measure. Perimeters, calibration of eye trackers, and automated refractors all require accurate eye fixation. Some devices for eye therapeutics also require the patient to fixate steadily on a small point in space for a certain period of time during which the eye does not move and is treated, for example treated with laser ablation therapy for laser vision correction, or laser treatment of structures within the eye.

Some (but not all) devices already have an optical subsystem that introduces a fixation target in the visual field of the test subject. For this purpose, a constant or blinking light is coupled into the subject's field of view, often by means of a beam splitter. The blinking light can be a low power laser (having the advantage of a small size, having a line spectrum, and suitable for manipulation by means of polarization optics), or, alternatively, an LED, which is simple to drive and cheaper.

Often times, the optics require that the target be monochromatic. The best case is a laser line, which makes it easy to separate the scanning beam from the target beam. In such cases, a low power laser is used, typically controlled by a square-wave generator of an appropriate frequency.

For young pediatric patients (ages under 5 years), who are typically less cooperative than older children and adults, unfortunately, a monotonously blinking target is not sufficient to attract attention steadily. This often makes it impossible to acquire data over time periods longer than a second for young pediatric patients. There thus remains a need for improved eye fixation systems.

### SUMMARY

An eye fixation system for attention-attracting fixation of a user's eye according to an embodiment of the current invention includes an audio system configured to play sound based on audio signals to be audible to a user so as to attract the user's attention, and a target display system configured to communicate with the audio system to receive the audio signals and to display an optical target to be viewed by the user. The target display system includes an optical modulator configured to modulate the optical target based on the audio signals.

A method of assisting a subject to fix at least one of his eyes in a predetermined alignment according to an embodiment of the current invention includes playing sound that is audible to the subject, displaying an optical target to be viewed by the subject synchronously with the sound such that the target is viewable by the subject while the sound is audible to the subject, and modulating the optical target based on the sound while the sound is being played.

A diagnostic system according to an embodiment of the current invention includes an eye fixation system for attention-attracting fixation of a user's eye in which the eye fixation system includes an audio system configured to play sound based on audio signals to be audible to a user so as to attract the user's attention, and a target display system configured to communicate with the audio system to receive the audio signals and to display an optical target to be viewed by the user. The target display system includes an optical modulator configured to modulate the optical target based on the audio signals.

### BRIEF DESCRIPTION OF THE DRAWINGS

Further objectives and advantages will become apparent from a consideration of the description, drawings, and examples.

FIG. 1 is a schematic illustration of an eye fixation system according to an embodiment of the current invention.

FIG. 2 is a schematic illustration of an eye fixation system according to another embodiment of the current invention.

FIG. 3 is a schematic illustration of a display screen that can be incorporated into the eye fixation systems of FIGS. 1 and 2 according to another embodiment of the current invention.

FIG. 4 is a schematic of a pulse width modulator according to an embodiment of the current invention.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without departing from the broad concepts of the current invention. All references cited anywhere in this specification, including the Background and Detailed Description sections, are incorporated by reference as if each had been individually incorporated.

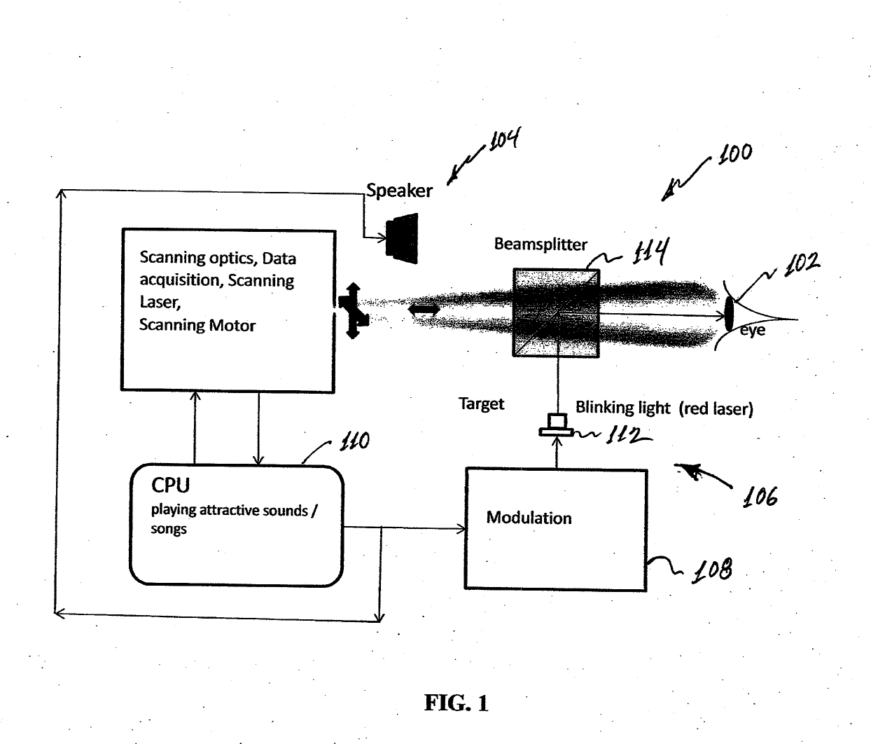

FIG. 1 is a schematic illustration of an eye fixation system 100 for attention-attracting fixation of a user's eye 102 according to an embodiment of the current invention. The eye fixation system 100 includes an audio system 104 configured to play sound based on audio signals to be audible to a user so as to attract the user's attention, and a target display system 106 configured to communicate with the audio system 104 to receive the audio signals and to display an optical target to be viewed by the user. The target display system 106 includes an optical modulator 108 configured to modulate the optical target based on the audio signals. In FIG. 1, the eye fixation system 100 is illustrated as being integrated into a diagnostic device. Both the eye fixation system 100 and devices that incorporate eye fixation system 100 are considered to be within the scope of the current invention.

In some embodiments, the eye fixation system 100 further includes a signal processing system 110 configured to communicate with the audio system 104 and the target display system 106. The signal processing system 110 can be configured to process the audio signals to provide a modulation signal to the optical modulator. The signal processing system 110 can be, or can be implemented on, a programmable computer, for example. However, the broad concepts of the current invention are not limited to that particular embodiment. Other embodiments can include signal processing systems implemented in hardware, such as, but not limited to, application-specific integrated circuits (ASICs) and/or field-programmable gate arrays (FPGAs), for example. Furthermore, the computer can be a localized, networked and/or distributed device, for example. In addition to including a central processing unit (CPU) and/or graphics processing unit (GPU), the computer can include memory and/or data storage, and/or input and output devices without limitation to any particular implementation. The sound signals and/or modulation signals can be generated in real-time and/or stored in memory and retrieved while in use.

In some embodiments, the target display system 106 includes a light emitting component 112. The light emitting component 112 can be, for example, a laser diode for some applications. There are many applications in which a laser is desirable to provide a substantially monochromatic, bright, and localized source of light. This can be useful in some applications in which a diagnostic device also uses a laser light source, but at a different emission wavelength, so that light from the two different sources can be projected simultaneously into the user's eye without interference. However, the broad concepts of the current invention are not limited to only one laser, and are not limited to only lasers. For example, the light emitting component 112 can be one or more light emitting diodes (LEDs) or even combinations of lasers and LEDs. For example, the light emitting component 112 can be a plurality of light emitting elements, such as, but not limited to, laser diodes and/or LEDs, or light emitting pixels within a liquid crystal display, each of which has a different central frequency of its emission spectrum. In some embodiments, the signal processing system 110 can be configured to provide modulation signals for each light emitting element.

In some embodiments, the eye fixation system 100 can further include additional optical components, depending on the particular application. For example, some embodiments of the target display system 106 can include a beam splitter 114 configured to project the optical target into the user's eye 102 while allowing light from a second light source to be projected into the user's eye 102 to substantially coincide with the optical target within the user's eye 102. The beam splitter can be, but is not limited to, prisms, dichroic mirrors, etc.

In some embodiments, the optical modulator 106 can be configured to perform pulse width modulation of light emitted by the light emitting component 112. Pulse width modulation can be thought of as light being “on” for a period of time, with “off” periods of time between pulses. The lengths of the on and off sequences depend on the audio signals. Pulse width modulation can be useful when lasers are used in order to be confident that safety thresholds on the light intensity are not exceeded. One should note, however, that the broad concepts of the current invention are not limited to only pulse width modulation.

In some embodiments, analog hardware that uses a sound wave, filters it to emphasize the frequencies of interest, finds the envelope of the sound curve, and modulates the intensity of the target can be used. For light levels much lower than the safety levels, amplitude modulation is acceptable. For light levels closer to the safety thresholds (as can be the case with laser targets), pulse-width modulation (PWM) can be used in order to keep peak amplitudes from reaching potentially harmful values. The pulse-width modulation frequency can be anywhere between 1 and 30 kHz, for example.

Some embodiments can include digital implementation of the modulation instead of, or in addition to, the analog implementations described above. A fast microprocessor or microcontroller, or a digital signal processor (DSP) can continuously perform Fast Fourier Transform (FFT) on the sound signal, with signal power in the spectral range of interest controlling modulation (amplitude or pulse-width), for example.

In some embodiments, analog hardware of the type described above can be used, but instead of modulating the intensity of the target, controlling the shape of a deformable lens to create a spot whose focus is pulsating with the sound coming through the speaker. The deformable lens can be a fast electrically tunable polymer lens, a liquid lens, or any other type. Loud sounds could cause the target to shrink and “focus” into a small spot, quiet periods could generate a blurry spot. Alternatively, the electrical signal could modulate the intensity of a diffuser, such as a liquid crystal diffuser.

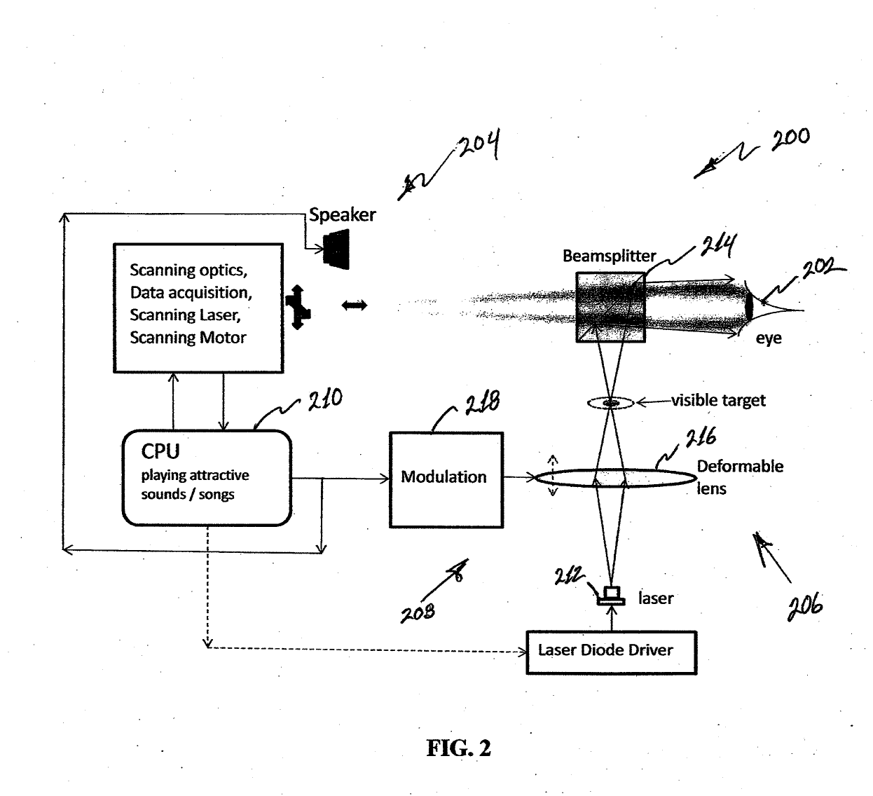

FIG. 2 is a schematic illustration of an eye fixation system 200 for attention-attracting fixation of a user's eye 202 according to another embodiment of the current invention. The eye fixation system 200 includes an audio system 204 configured to play sound based on audio signals to be audible to a user so as to attract the user's attention, and a target display system 206 configured to communicate with the audio system 204 to receive the audio signals and to display an optical target to be viewed by the user. The target display system 206 includes an optical modulator 208 configured to modulate the optical target based on the audio signals. Similar to FIG. 1, the eye fixation system 200 is illustrated as being integrated into a diagnostic device. Both the eye fixation system 200 and devices that incorporate eye fixation system 200 are considered to be within the scope of the current invention.

In some embodiments, the eye fixation system 200 further includes a signal processing system 210 configured to communicate with the audio system 204 and the target display system 206. The signal processing system 210 can be configured to process the audio signals to provide a modulation signal to the optical modulator. Similar to the embodiment of FIG. 1, the signal processing system 210 can be, or can be implemented on, a programmable computer, for example. However, the broad concepts of the current invention are not limited to that particular embodiment. Other embodiments can include signal processing systems implemented in hardware, such as, but not limited to, ASICs and/or FPGAs, for example. Furthermore, the computer can be a localized, networked and/or distributed device, for example. In addition to including a CPU and/or GPU, the computer can include memory and/or data storage, and/or input and output devices without limitation to any particular implementation. The sound signals and/or modulation signals can be generated in real-time and/or stored in memory and retrieved while in use.

In some embodiments, the target display system 106 includes a light emitting component 212. The light emitting component 112 can be, for example, a laser diode for some applications. There are many applications in which a laser is desirable to provide a substantially monochromatic, bright, and localized source of light. This can be useful in some applications in which a diagnostic device also uses a laser light source, but at a different emission wavelength, so that light from the two different sources can be projected simultaneously into the user's eye without interference. However, the broad concepts of the current invention are not limited to only one laser, and are not limited to only lasers. For example, the light emitting component 212 can be one or more light emitting diodes (LEDs) or even combinations of lasers and LEDs. For example, the light emitting component 212 can be a plurality of light emitting elements, such as, but not limited to, laser diodes and/or LEDs, or light emitting pixels within a liquid crystal display, each of which has a different central frequency of its emission spectrum. In some embodiments, the signal processing system 210 can be configured to provide modulation signals for each light emitting element.

In some embodiments, the eye fixation system 200 can further include additional optical components, depending on the particular application. For example, some embodiments of the target display system 206 can include a beam splitter 214 configured to project the optical target into the user's eye 202 while allowing light from a second light source to be projected into the user's eye 202 to substantially coincide with the optical target within the user's eye 202. The beam splitter can be, but is not limited to, prisms, dichroic mirrors, etc.

The optical modulator 208 includes a deformable lens 216 and an actuation unit 218. In this embodiment, the target seems to be varying between a wide, blurry spot and a small, well-focused spot. The modulation is derived from the audio such that the variations in the focus of the spot are correlated with the audio that is being played.

The curvature of the lens' shape, or the effective refractive index of the lens material, is adjusted by applying electrical current such that the focal length can be precisely tuned. For the sound frequencies of interest, the eye sees a shape close to a point during loud portions of the sound, and a blurred spot during quiet sound, or vice-versa. The target is perceived as a pulsating spot, being focused and defocused synchronously with the sound.

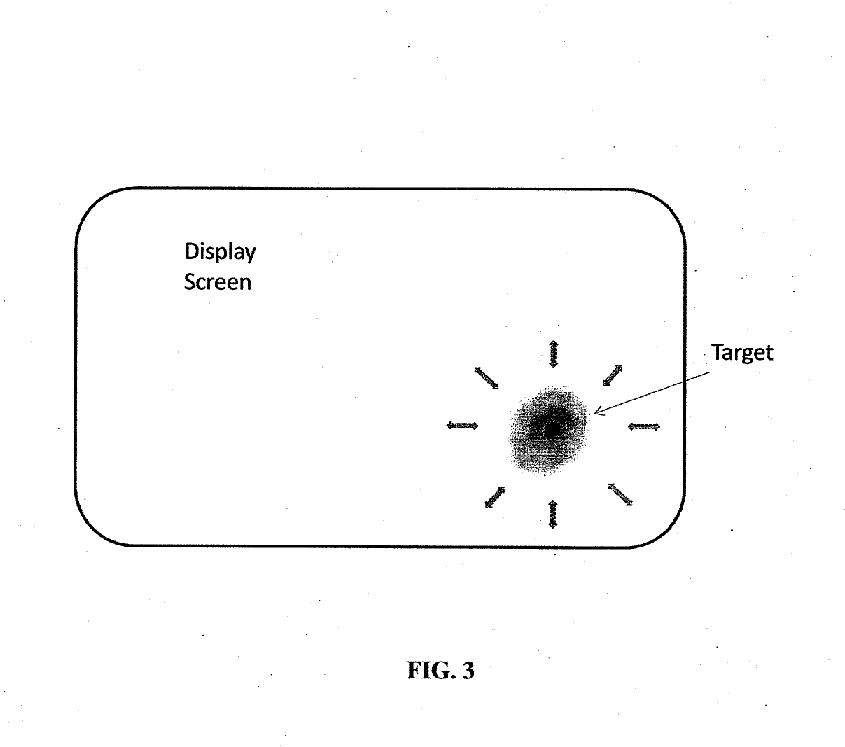

A further embodiment is similar to that of FIG. 2, except the deformable lens and laser diode are replaced by a display screen. For example, the display screen can be a liquid crystal display (LCD), a light emitting diode (LED) display, an organic light emitting diode (OLED) display, or any other screen type display, where the pixels may be self-luminous or including backlighting, may contain optical switches and mirrors to redirect or modulate light beams, may be a digital micromirror device (DMD), a digital light processing (DLP) projector device, a liquid crystal on silicon (LCoS) device, or any other matrix projection device for front or rear projection. A schematic example of a display screen is shown in FIG. 3. This can be used to change the brightness of a spot and/or a multi-pixel appearance based on the audio that is being played. The target spot may vary in size, intensity, color, shape, texture, glow, shadow, etc., based on the current sound level, frequency content, attack, or other audio parameters.

One can see that a wide variety of types of modulation can be included according to various embodiments of the current invention. The target can be point-like, or even have some spatial extent such as to appear as an image or pattern. The modulation can modulate one or more properties of the light. This can be, but is not limited to, intensity, focus, size, shape or pattern, color, polarization, etc. The target thus appears to have some type of time-varying behavior to the user. The time varying behavior appears to be correlated to, or generated by, the audio due to the modulation using the audio signal.

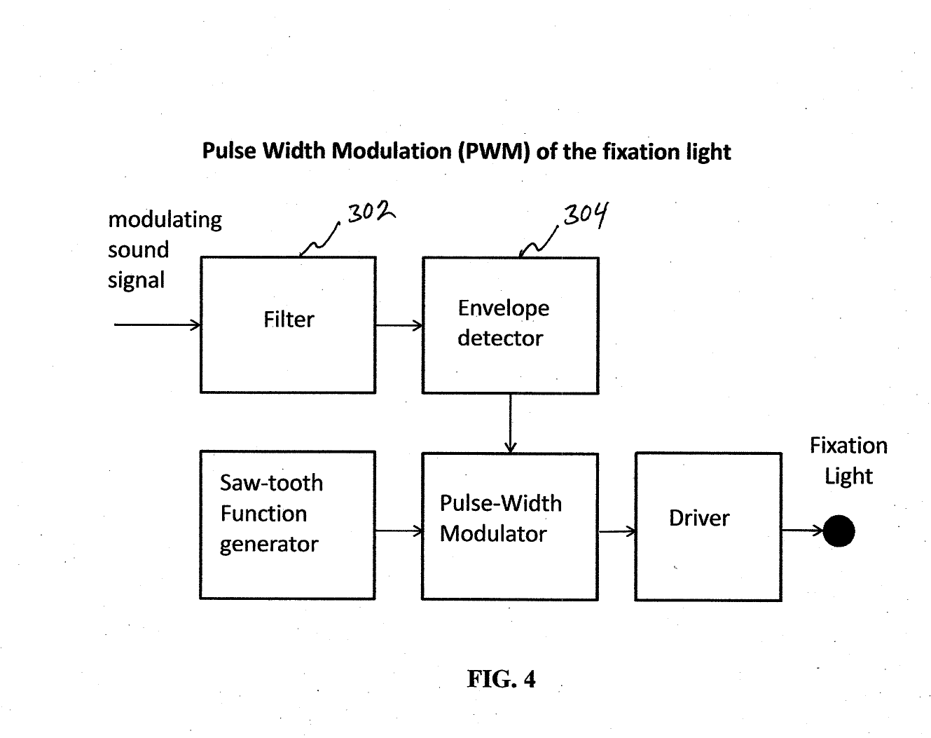

FIG. 4 is a schematic of an implementation of pulse width modulation according to an embodiment of the current invention. As noted above, it can be implemented on a programmed computer and/or in dedicated hardware. In this example, the signal processing system (110, 210) can include a band-pass filter 302 configured to receive and filter the audio signal to provide a filtered audio signal, and an envelope detector 304 configured to process the filtered audio signals to provide the modulation signal to the optical modulator (108, 208).

In some embodiments, the components illustrated in FIG. 4 up to the driver can be implemented entirely digitally. The filter can be a band-pass filter whose central frequency depends on the type of the modulating sound. For music, a suitable central frequency may be 500 Hz, −3 dB at 150 Hz and 850 Hz, respectively. For voice and laughter, a suitable central frequency may be 1500 Hz, −3 dB at 1000 Hz and 2000 Hz, respectively. For computer gaming sounds, the filter settings may vary, depending on the type of sounds used. For example, an acceptable filter setting may be one of central frequency 800 Hz, −3 dB at 500 Hz and 1200 Hz, respectively. One approach would be to perform FFT on the modulating sound, then define the central frequency at the median of the spectrum, and apply digital filtering.

For the case of color modulation using a multicolor LED (say, 3 LEDs in one—i.e. R,G,B), the above pulse width modulation can be applied separately for each of the three LEDs, for example. Color separation may be done on a spectral basis (the way it was done in the 80's in virtually all disco's), or on amplitude basis using a window comparator. Both of these techniques can be implemented either in analog, or in a digital manner.

In the case of modulating patterns on an LCD or LED or OLED (or other digital) display, the modulation by sound may be done in real time, while the patterns are being generated. Alternatively, it may be done off-line by previously generating patterns that change synchronously with the music, and placing everything in a video file (with sound). The intensity of the pattern may be best controlled by changing the number of pixels involved, as well as changing the color and the contrast.

In operation, some embodiments use a combination of sound and modulated low power laser light to attract the attention of a young child and to serve as a fixation target. A computer (CPU) controls the scanning optics and the data acquisition, and simultaneously plays sounds or songs attractive to the subjects. The sound wave is also fed to a modulation circuit which controls the target laser. The target laser is introduced into the eye's visual field by means of a beam splitter, which can be polarizing or non-polarizing, depending on the scanning method. The target laser and the scanning laser are of different wavelengths, so that they do not interfere with each other. The eye fixates on the target laser over a longer period of time, during which the retina does not move and is efficiently scanned. The speaker is positioned in a way that the subject perceives it as coinciding with the target. The target laser is modulated and is “pulsating” synchronously with the sound. This strongly enhances the attention attraction ability and locks fixation on the target. Modulation can be of amplitude or pulse-width type, or even other types of modulation.

Another embodiment of the current invention is directed to a method of assisting a subject to fix at least one of his eyes in a predetermined alignment. The embodiment includes playing sound that is audible to said subject, displaying an optical target to be viewed by the subject synchronously with the sound such that the target is viewable by the subject while the sound is audible to the subject, and modulating the optical target based on the sound while the sound is being played.

Another embodiment of the current invention is directed to a diagnostic system that includes an eye fixation system for attention-attracting fixation of a user's eye. The eye fixation system can be any eye fixation system according to embodiments of the current invention, depending on the particular application. The diagnostic system can be, but is not limited to, a scanning laser ophthalmoscope, an optical coherence tomography (OCT) system, a retinal tomograph, a scanning laser polarimeter, a retinal birefringence scanner, a fundus camera, and others. Other aspects of the current invention can include behavioral or psychological tests where deviations from steady fixation on a target are used as a differentiating measure. Still other aspects of the current invention can include fixation systems used in perimeters, eye trackers, automated refractors, etc. Still other aspects of the current invention are applicable to fixation systems used in devices for eye treatment such as laser ablation therapy for laser vision correction, or laser treatment of structures within the eye.

The embodiments illustrated and discussed in this specification are intended only to teach those skilled in the art how to make and use the invention. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

We claim:

1. An eye fixation system for attention-attracting fixation of a user's eye, comprising:
an audio system configured to play sound based on audio signals to be audible to a user so as to attract said user's attention; and
a target display system configured to communicate with said audio system to receive said audio signals and to display an optical target to be viewed by said user,
wherein said target display system comprises an optical modulator configured to modulate said optical target based on said audio signals.

2. An eye fixation system according to claim 1, further comprising a signal processing system configured to communicate with said audio system and said target display system,
wherein said signal processing system is configured to process said audio signals to provide a modulation signal to said optical modulator.

3. An eye fixation system according to claim 2, wherein said target display system comprises a laser.

4. An eye fixation system according to claim 2, wherein said target display system comprises a light emitting diode.

5. An eye fixation system according to claim 2, wherein said target display system comprises a plurality of light emitting elements, each with a different central frequency of a corresponding emission spectrum.

6. An eye fixation system according to claim 5, wherein said signal processing system is configured to process said audio signals to provide to said optical modulator a modulation signal for each of said plurality of light emitting elements.

7. An eye fixation system according to claim 6, wherein said plurality of light emitting elements are at least one of light emitting diodes or lasers.

8. An eye fixation system according to claim 3, wherein said optical modulator of said target display system is configured to perform pulse width modulation of light emitted by said laser.

9. An eye fixation system according to claim 2, wherein said signal processing system comprises a band-pass filter configured to receive and filter said audio signal to provide a filtered audio signal, and
wherein said signal processing system further comprises an envelope detector configured to process said filtered audio signals to provide said modulation signal to said optical modulator.

10. An eye fixation system according to claim 9, wherein said modulation signal is a pulse width modulation signal.

11. An eye fixation system according to claim 9, wherein said band-pass filter has a center frequency and a band width selected according to a type of sound to be played by said audio system.

12. An eye fixation system according to claim 11, wherein said type of sound to be played by said audio system is at least one of music, voice and laughter, or computer gaming sounds.

13. An eye fixation system according to claim 3, wherein said optical modulator of said target display system further comprises a deformable lens such that a focus of light emitted from said laser is modulated using said audio signals.

14. An eye fixation system according to claim 3, wherein said optical modulator of said target display system further comprises an electronically driven diffuser such that a focus of light emitted from said laser is modulated using said audio signals.

15. An eye fixation system according to claim 3, wherein said target display system further comprises a beam splitter configured to project said optical target into said user's eye while allowing light from a second light source to be projected into said user's eye to substantially coincide with said optical target within said user's eye.

16. An eye fixation system according to claim 15, wherein said second light source comprises a second laser having a different emission wavelength from an emission wavelength from said laser of said target display system.

17. An eye fixation system according to claim 2, wherein said target display system comprises a display screen.

18. An eye fixation system according to claim 17, wherein said display screen is one of a liquid crystal display, a light emitting diode display, or an organic light emitting diode display.

19. An eye fixation system according to claim 17, wherein said display screen comprises self-luminous pixels.

20. An eye fixation system according to claim 17, wherein said display screen comprises backlighting.

21. A method of assisting a subject to fix at least one of his eyes in a predetermined alignment, comprising:
playing sound that is audible to said subject;
displaying an optical target to be viewed by said subject synchronously with said sound such that said target is viewable by said subject while said sound is audible to said subject; and
modulating said optical target based on said sound while said sound is being played.

22. A diagnostic system comprising an eye fixation system for attention-attracting fixation of a user's eye, said eye fixation system comprising:
an audio system configured to play sound based on audio signals to be audible to a user so as to attract said user's attention; and
a target display system configured to communicate with said audio system to receive said audio signals and to display an optical target to be viewed by said user,
wherein said target display system comprises an optical modulator configured to modulate said optical target based on said audio signals.
